(19) United States
(12) Patent Application Publication
(10) Pub. No.: US 2011/0046190 A1
(43) Pub. Date: Feb. 24, 2011

(54) COMBINATIONS COMPRISING A SELECTIVE CYCLOOXYGENASE-2 INHIBITOR

(76) Inventors: Ying-Nan Pan CHEN, Parsippany, NJ (US); Peter Lassota, Succasunna, NJ (US); Alexander Wallace Wood, Ho Ho Kus, NJ (US)

Correspondence Address: NOVARTIS;CORPORATE INTELLECTUAL PROPERTY, ONE HEALTH PLAZA 101/2, EAST HANOVER, NJ, 07936-1080, US

(21) Appl. No.: 12/938,868

(22) Filed: Nov. 3, 2010

Related U.S. Application Data
(63) Continuation of application No. 10/493,297, filed on Oct. 8, 2004.
(60) Provisional application No. 60/344,734, filed on Oct. 25, 2001.
(60) Provisional application No. 60/344,735, filed on Oct. 25, 2001.
(60) Provisional application No. 60/336,033, filed on Nov. 15, 2001.

(51) Int. Cl. A61K 31/427; A61P 35/00; A61P 1/00

(52) U.S. Cl. 514/365

(57) ABSTRACT

A combination therapy for treating patients suffering from pre-malignant colon lesions (e.g. polyps) and colon cancer, as well as other malignancies, is disclosed. The patient is treated concurrently with a cycloocygenase-2 inhibitor and at least one compound selected from the group consisting of a microtubule interfering agent, an epithelial growth factor receptor tyrosine protein kinase inhibitor and a vascular endothelial growth factor receptor tyrosine kinase inhibitor.

The invention relates to a method of preventing or treating pre-malignant colon lesions (e.g. polyps) and colon cancer, as well as other malignancies in a warm-blooded animal, especially a mammal, particularly a human, with a combination of pharmaceutical agents which comprises (a) a selective cyclooxygenase-2 inhibitor (“COX-2 inhibitor”) and (b) at least one compound selected from the group consisting of a microtubule interfering agent (“MIA”), a non-covalent epithelial growth factor receptor tyrosine protein kinase inhibitor (“EGFR inhibitor”) and a vascular endothelial growth factor receptor tyrosine kinase inhibitor (“VEGF inhibitor”). The invention further relates to pharmaceutical compositions comprising (a) COX-2 inhibitor and (b) at least one compound selected from the group consisting of an MIA, an EGFR inhibitor and a VEGF inhibitor and (c) a pharmaceutically acceptable carrier. The present invention further relates to a commercial package or product comprising (a) a pharmaceutical formulation of a COX-2 inhibitor and (b) at least one pharmaceutical formulation of a compound selected from the group consisting of an MIA, an EGFR inhibitor and an VEGF inhibitor for simultaneous, concurrent, separate or sequential use.

Non-steroidal antiinflammatory agents are known to block prostaglandin synthesis by inhibition of the enzyme cyclooxygenase. Cyclooxygenase is now known to comprise a constitutive isoform, cyclooxygenase-1 (“COX-1”), and an inducible isoform, cyclooxygenase-2 (“COX-2”).

COX-2 inhibitors are known in the art as compounds that selectively inhibit cyclooxygenase-2 without appreciable inhibition of cyclooxygenase-1. Methods of measuring the inhibition of cyclooxygenase-1 and -2 are known in the art.

Without being bound to any particular theory, it is postulated that the improved efficacy seen with a combination comprising a COX-2 inhibitor and a VEGF inhibitor is based on the following findings. Cyclooxygenase-2 is expressed in endothelial cells during tumor neovascularization as well as in epithelial cancer cells present in human colon, breast, prostate and lung tissues. It is known that prostaglandin E2 or I2 generated by COX-2 induces VEGF receptors in the endothelial cells and accelerates angiogenesis. The formation of new blood vessels to provide nutrients is a major requirement for the growth of solid tumors. VEGF inhibitors also inhibit neovascularization by inhibition of vascular endothelial growth factor receptors. Thus, the combination of agents synergistically inhibit neovascularization and tumor activity by both reducing the number of vascular endothelial growth factor receptors and by inhibiting those receptors that are present.

Of the known COX-2 inhibitors, the 5-alkyl substituted 2-arylaminophenylacetic acids and derivatives are especially useful in the present invention. Such compounds, their use, preparation and galenical formulations comprising such compounds are disclosed in U.S. Pat. No. 6,291,523, which is here incorporated by reference.

Useful COX-2 inhibitors disclosed in U.S. Pat. No. 6,291,523 are described by formula I

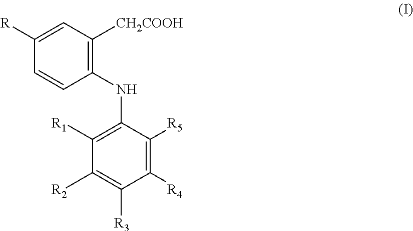

wherein
R is methyl or ethyl;
R₁ is chloro or fluoro;
R₂ is hydrogen or fluoro;
R₃ is hydrogen, fluoro, chloro, methyl, ethyl, methoxy, ethoxy or hydroxy;
R₄ is hydrogen or fluoro; and
R₅ is chloro, fluoro, trifluoromethyl or methyl;
pharmaceutically acceptable salts or solvates thereof; and
pharmaceutically acceptable prodrug esters thereof.

A particular embodiment of the invention relates to the compounds of formula I wherein R is methyl or ethyl; R₁ is chloro or fluoro; R₂ is hydrogen; R₃ is hydrogen, fluoro, chloro, methyl or hydroxy; R₄ is hydrogen; and R₅ is chloro, fluoro or methyl; pharmaceutically acceptable salts thereof; and pharmaceutically acceptable prodrug esters thereof.

A preferred embodiment relates to the compounds of formula I wherein R is methyl or ethyl; R₁ is fluoro; R₂ is hydrogen; R₃ is hydrogen, fluoro or hydroxy; R₄ is hydrogen; and R₅ is chloro; pharmaceutically acceptable salts thereof; and pharmaceutically acceptable prodrug esters thereof.

Another preferred embodiment of the invention relates to compound of formula I wherein R is ethyl or methyl; R₁ is fluoro; R₂ is hydrogen or fluoro; R₃ is hydrogen, fluoro, ethoxy or hydroxy; R₄ is hydrogen or fluoro; and R₅ is chloro, fluoro or methyl; pharmaceutically acceptable salts thereof; and pharmaceutically acceptable prodrug esters thereof.

Further preferred are said compounds wherein R is methyl or ethyl; R₁ is fluoro; R₂-R₄ are hydrogen or fluoro; and R₅ is chloro or fluoro; pharmaceutically acceptable salts thereof; and pharmaceutically acceptable prodrug esters thereof.

A further embodiment of the invention relates to the compounds of formula I wherein R is methyl or ethyl; R₁ is fluoro; R₂ is fluoro; R₃ is hydrogen, ethoxy or hydroxy; R₄ is fluoro; and R₅ is fluoro; pharmaceutically acceptable salts thereof; and pharmaceutically acceptable prodrug esters thereof.

Another preferred embodiment of the invention relates to the compounds of formula I wherein R is methyl; R₁ is fluoro; R₂ is hydrogen; R₃ is hydrogen or fluoro; R₄ is hydrogen; and R₅ is chloro; pharmaceutically acceptable salts thereof; and pharmaceutically acceptable prodrug esters thereof.

Particular embodiments of the invention relate to compounds of formula I
(a) wherein R is methyl; R₁ is fluoro; R₂ is hydrogen; R₃ is hydrogen; R₄ is hydrogen; and R₅ is chloro; pharmaceutically acceptable salts thereof; and pharmaceutically acceptable prodrug esters thereof;
(b) wherein R is methyl; R₁ is fluoro; R₂ is hydrogen; R₃ is fluoro; R₄ is hydrogen; and R₅ is chloro; pharmaceutically acceptable salts thereof; and pharmaceutically acceptable prodrug esters thereof;
(c) wherein R is ethyl; R₁ is fluoro; R₂ is fluoro; R₃ is hydrogen; R₄ is fluoro; and R₅ is fluoro; pharmaceutically acceptable salts thereof; and pharmaceutically acceptable prodrug esters thereof; and
(d) wherein R is ethyl; R₁ is chloro; R₂ is hydrogen; R₃ is chloro; R₄ is hydrogen; and R₅ is methyl; pharmaceutically acceptable salts thereof; and pharmaceutically acceptable prodrug esters thereof.

The general definitions used herein have the following meaning within the scope of the present invention.

Pharmaceutically acceptable prodrug esters are ester derivatives which are convertible by solvolysis or under physiological conditions to the free carboxylic acids of formula I. Such esters are e.g. lower alkyl esters (such as the methyl or ethyl ester), carboxy-lower alkyl esters such as the carboxymethyl ester, nitrooxy-lower alkyl esters (such as the 4-nitro-oxybutyl ester), and the like. Preferred are the 5-alkyl substituted 2-arylaminophenyl-acetoxyacetic acids of formula Ia

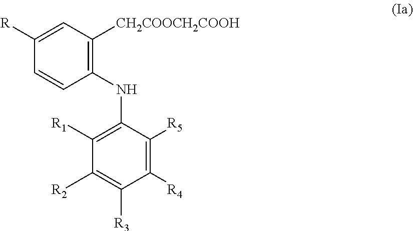

wherein R and R₁-R₅ have meaning as defined hereinabove for compounds of formula I; and pharmaceutically acceptable salts thereof.

Pharmaceutically acceptable salts represent metal salts, such as alkaline metal salts, e.g. sodium, potassium, magnesium or calcium salts, as well as ammonium salts, which are formed e.g. with ammonia and mono- or di-alkylamines, such as diethylammonium salts, and with amino acids, such as arginine and histidine salts.

The compound 5-methyl-2-(2′-chloro-6′-fluoro-anilino)-phenyl acetic acid, as well as its pharmaceutically acceptable salts, is an especially useful COX-2 inhibitor for use in the present invention.

Therefore, the present invention relates to a method for the prevention or treatment of pre-malignant colon lesions or a colon cancer, or other malignancy, in a mammal, which comprises treating the mammal concurrently with a combination of (a) a selective COX-2 inhibitor and (b) a VEGF inhibitor.

Furthermore, the present invention relates to a method for the prevention or treatment of pre-malignant colon lesions or a colon cancer or other malignancy in a mammal, which comprises treating the mammal concurrently with a combination of (a) a selective COX-2 inhibitor of the formula (I) wherein R is methyl or ethyl; R₁ is chloro or fluoro; R₂ is hydrogen or fluoro; R₃ is hydrogen, fluoro, chloro, methyl, ethyl, methoxy, ethoxy or hydroxy; R₄ is hydrogen or fluoro; and R₅ is chloro, fluoro, trifluoromethyl or methyl; pharmaceutically acceptable salts or solvates thereof; and pharmaceutically acceptable prodrug esters thereof; and (b) at least one compound selected from the group consisting of an MIA, an EGFR inhibitor and a VEGF inhibitor.

In particular, the present invention relates to a combination which comprises (a) a selective COX-2 inhibitor and (b) a VEGF inhibitor, in which the active ingredients (a) and (b) are present in each case in free form or in the form of a pharmaceutically acceptable salt and optionally at least one pharmaceutically acceptable carrier; for simultaneous, separate or sequential use.

Moreover, the present invention relates to a combination which comprises (a) a selective COX-2 inhibitor of the formula (I) wherein R is methyl or ethyl; R₁ is chloro or fluoro; R₂ is hydrogen or fluoro; R₃ is hydrogen, fluoro, chloro, methyl, ethyl, methoxy, ethoxy or hydroxy; R₄ is hydrogen or fluoro; and R₅ is chloro, fluoro, trifluoromethyl or methyl; pharmaceutically acceptable salts or solvates thereof; and pharmaceutically acceptable prodrug esters thereof; and (b) at least one compound selected from the group consisting of an MIA, a non-covalent EGF inhibitor and a VEGF inhibitor, in which the active ingredients (a) and (b) are present in each case in free form or in the form of a pharmaceutically acceptable salt and optionally at least one pharmaceutically acceptable carrier; for simultaneous, separate or sequential use.

Preferred combinations are in particular those wherein
the selective COX-2 inhibitor of the formula (I) is 5-methyl-2-(2′-chloro-6′-fluoro-anilino)-phenyl acetic acid or a pharmaceutically acceptable salt thereof,
the combination partner (b) is an microtubule interfering agent selected from colchicine, a podophyllotoxin, a taxane, a discodermolide compound, a vinca alkaloid or an epothilone, in particular from paclitaxel, docetaxel, epothilone B and (+)-discodermolide,
(a) the COX-2 inhibitor is selected from the group consisting of 5-methyl-2-(2′-chloro-6′-fluoro-anilino)-phenyl acetic acid, or a pharmaceutically acceptable salt thereof, and (b) is an VEGF inhibitor selected from the group consisting of 1-(4-chloroanilino)-4-(4-pyridylmethyl)phthalazine, or a pharmaceutically acceptable salt thereof,

The combinations disclosed herein are suitable in particular for the use in the treatment of a proliferative disease and for use in the prevention or treatment of pre-malignant colon lesions or colon cancer.

According to the present invention, a patient is treated, e.g., concurrently with a COX-2 inhibitor and at least one compound selected from the group consisting of an MIA, an EGFR inhibitor and a VEGF inhibitor in order to prevent or treat pre-malignant colon lesions, such as polyps, or colon cancer, or another malignancy each according to a dosage schedule that is appropriate for the individual agent. For example, the COX-2 inhibitor may be administered once or more daily and an MIA may be administered once daily, on alternate days or on some other schedule—as is appropriate for the MIA agent when used without the COX-2 inhibitor.

MIA compounds are known and clinically used for the treatment of cancer. Such compounds include colchicine, podophyllotoxins, such as etoposide and teniposide, taxanes, such as paclitaxel and docetaxel, discodermolide compounds, which includes (+)-discodermolide and analogs and derivatives of (+)-discodermolide, vinca alkaloids, such as vinblastin, especially vinblastine sulfate, vincristine, especially vincristine sulfate, and vinorelbine, and epothilones, such as epothilones A, B, C and D, as well as analogs and derivatives thereof, for example the compounds disclosed in WO 99/02514, particularly [1 S-[1R, 3R(E), 7R, 10S, 11R, 12R, 16S]]-7,11-dihydroxy-8,8,10,12,16-pentamethyl-3-[1-methyl-2-(2-methyl-4-thiazolyl)ethenyl]-4-aza-17-bicyclo[14.1.0]-heptadecane-5,9-dione (example 3). Vinblastine sulfate can be administered, e.g., in the form as it is marketed, e.g. under the trademark VINBLASTIN R.P.™. Vincristine sulfate can be administered, e.g., in the form as it is marketed, e.g. under the trademark FARMISTIN™. Discodermolide can be obtained, e.g., as disclosed in U.S. Pat. Nos. 4,939,168 and 5,618,487 to Harbor Branch Oceanographic Institute or by chemical synthesis as described, for example, in GB 2280677, WO 98/24429 and U.S. Pat. Nos. 5,789,605 and 6,031,133, which are here incorporated by reference. Etoposide can be administered, e.g., in the form as it is marketed, e.g. under the trademark ETOPOPHOS™. Teniposide can be administered, e.g., in the form as it is marketed, e.g. under the trademark VM 26-BRISTOL™.

Discodermolide, as well as its analogs and derivatives, are especially useful MIA compounds. Discodermolide and its preparation are known in the art. The preparation of analogs and derivatives has also been reported in the literature.

Epothilones that can be used in the present invention are described by formula (II),

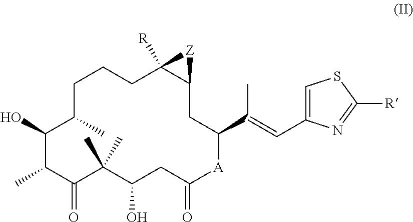

wherein A represents O or NR_{N}, wherein R_{N} is hydrogen or lower alkyl, R is hydrogen or lower alkyl, R′ is methyl, methoxy, ethoxy, amino, methylamino, dimethylamino or methylthio, and Z is O or a bond.

Unless stated otherwise, in the present disclosure organic radicals and compounds designated “lower” contain not more than 7, preferably not more than 4, carbon atoms.

A compound of formula II wherein A represents O, R is hydrogen, R′ is methyl and Z is O is known as epothilone A; a compound of formula II wherein A represents O, R is methyl, R′ is methyl and Z is O is known as epothilone B; a compound of formula II wherein A represents O, R is hydrogen, R′ is methyl and Z is a bond is known as epothilone C; a compound of formula II wherein A represents O, R is methyl, R′ is methyl and Z is a bond is known as epothilone D.

Epothilone derivatives of formula II wherein A represents O or NR_{N}, wherein R_{N} is hydrogen or lower alkyl, R is hydrogen or lower alkyl, R′ is methyl and Z is O or a bond, and methods for the preparation of such epothilone derivatives are in particular generically and specifically disclosed in the patents and patent applications WO 93/10121, U.S. Pat. No. 6,194,181, WO 98/25929, WO 98/08849, WO 99/43653, WO 98/22461 and WO 00/31247 in each case in particular in the compound claims and the final products of the working examples, the subject-matter of which is hereby incorporated into the present application by reference to this publications. Comprised are likewise the corresponding stereoisomers as well as the corresponding crystal modifications, e.g. solvates and polymorphs, which are disclosed therein. Epothilone derivatives of formula II, especially epothilone B, can be administered as part of pharmaceutical compositions which are disclosed in WO 99/39694.

Epothilone derivatives of formula II wherein A represents O or NR_{N}, wherein R_{N} is hydrogen or lower alkyl, R is hydrogen or lower alkyl, R′ is methoxy, ethoxy, amino, methylamino, dimethylamino or methylthio, and Z is O or a bond, and methods for the preparation and administration of such epothilone derivatives are in particular generically and specifically disclosed in the patent application WO99/67252, which is hereby incorporated by reference into the present application. Comprised are likewise the corresponding stereoisomers as well as the corresponding crystal modifications, e.g. solvates and polymorphs, which are disclosed therein.

The transformation of epothilone B to the corresponding lactam is disclosed in Scheme 21 (page 31, 32) and Example 3 of WO 99/02514 (pages 48-50). The transformation of a compound of formula II which is different from epothilone B into the corresponding lactam can be accomplished analogously. Corresponding epothilone derivatives of formula II wherein R_{N} is lower alkyl can be prepared by methods known in the art such as a reductive alkylation reaction starting from the epothilone derivative wherein R_{N} is hydrogen.

It is known that MIA compounds such as paclitaxel, discodermolide, colchicine and vinblastine increase levels of prostaglandin E2 through upregulation of COX-2. Without being bound to any particular hypothesis, it is postulated that this effect may partially counteract the antiproliferative effects of MIA compounds. Thus, enhancement of the anti-tumor activity of the MIA by inhibition of COX-2 may be basis for the improved effect observed when, according to the present invention, a COX-2 inhibitor is added to a cancer treatment regimen with a MIA. As an added benefit, the COX-2 inhibitor may help manage cancer-related pain and inflammation.

In one aspect, the present invention relates to a method for the prevention of treatment of pre-malignant colon lesions (e.g. polyps), and colon cancers and other malignancies in a mammal, preferably a human patient, which comprises treating the patient concurrently with a combination of (a) a COX-2 inhibitor of U.S. Pat. No. 6,291,523 and (b) a MIA.

In addition to the prevention and treatment of pre-malignant colon lesions (e.g. polyps) and colon cancer, the inventive combination therapy has utility for the treatment of “other malignancies”, which is hereby defined as a malignancy that is susceptible to treatment with an MIA compound, for example, breast cancer, lung cancer, ovarian cancer, lymphoma, head and neck cancer and cancer of the esophagus, stomach, bladder, prostrate, uterus and cervix.

Most preferably, the present invention relates to a method for the prevention or treatment of pre-malignant colon lesions, colon cancer or another malignancy in a human patient, which comprises treating the patient concurrently with a combination of (a) a COX-2 inhibitor selected from the group consisting of 5-methyl-2-(2′-chloro-6′-fluoro-anilino)-phenyl acetic acid, or a pharmaceutically acceptable salt thereof, and (b) an MIA selected from the group consisting of colchicine, a podophyllotoxin, a taxane, a discodermolide compound, a vinca alkaloid and an epothilone. Especially, the MIA is a taxane, an epothilone or a discodermolide compound, preferably the MIA is paclitaxel, docetaxel, epothilone B or (+)-discodermolide, especially (+)-discodermolide. In a specific embodiment, the inventive method is a method for the prevention or treatment of colon cancer. In another embodiment, the inventive method is a method for the treatment of other malignancies as described above.

Preferably, treatment using a COX-2 inhibitor within one of the preferred classes disclosed in U.S. Pat. No. 6,291,523 is combined with treatment using an MIA selected from colchicine, a podophyllotoxin, a taxane, a discodermolide compound, a vinca alkaloid or an epothilone. Preferably, the MIA is paclitaxel, docetaxel, epothilone B or discodermolide.

EGFR inhibitors and their use as agents for the treatment of cancer are also known in the art. Non-covalent EGFR inhibitors useful in the present invention especially include 7H-pyrrolo{2,3-d}pyrimidine derivatives, such as those described in U.S. Pat. No. 6,140,332, which is here incorporated by reference. Salts and solvates of the 7H-pyrrolo{2,3-d}pyrimidine derivatives are included in the EGFR inhibitors useful in the present invention.

The compound (R)-4-(4-((1-phenylethyl)amino)-7H-pyrrolo(2,3-d)pyrimidin-6-yl)-phenol, or a pharmaceutically acceptable salt thereof, is the preferred 7H-pyrrolo{2,3-d}pyrimidine derivative for use in the present invention.

Hence, in another aspect, the present invention relates to a method for the prevention of treatment of pre-malignant colon lesions (e.g. polyps) and colon cancers in a human patient, which comprises treating the patient concurrently with a combination of (a) a COX-2 inhibitor of U.S. Pat. No. 6,291,523 and (b) a non-covalent EGFR inhibitor of U.S. Pat. No. 6,140,332.

In one aspect, treatment using a COX-2 inhibitor within one of the preferred classes disclosed in U.S. Pat. No. 6,291,523 is combined with treatment using an EGFR inhibitor within one of the preferred classes disclosed in U.S. Pat. No. 6,140,332.

In another aspect, treatment using a COX-2 inhibitor within one of the preferred classes disclosed in U.S. Pat. No. 6,291,523 is combined with treatment using an EGFR inhibitor selected from the group consisting of (R)-4-(4-((1-phenylethyl)amino)-7H-pyrrolo(2,3-d)pyrimidin-6-yl)-phenol, or a pharmaceutically acceptable salt thereof.

In a different aspect, treatment using a COX-2 inhibitor selected from the group consisting of 5-methyl-2-(2′-chloro-6′-fluoro-anilino)-phenyl acetic acid, or a pharmaceutically acceptable salt thereof, is combined with treatment using an EGFR inhibitor within one of the preferred classes disclosed in U.S. Pat. No. 6,140,332.

In addition to the prevention and treatment of pre-malignant colon lesions (e.g. polyps) and colon cancer, the inventive combination therapy has utility for the treatment of “other malignancies”, which is hereby defined as a malignancy that is susceptible to treatment with an EGFR inhibitor, for example, breast cancer, lung cancer, ovarian cancer, lymphoma, head and neck cancer and cancer of the esophagus, stomach, bladder, prostrate, uterus and cervix.

According to the present invention, a patient is treated concurrently with a COX-2 inhibitor and an EGFR inhibitor in order to prevent or treat pre-malignant colon lesions, such as polyps, or colon cancer, or another malignancy, each according to a dosage schedule that is appropriate for the individual agent. For example, the COX-2 inhibitor may be administered once or more daily and the EGFR inhibitor may be administered once daily, on alternate days or on some other schedule.

VEGF inhibitors and their use for the treatment of cancer are known in the art. Important VEGF inhibitors are the 4-pyridylmethyl-phthalazine derivatives that are described in U.S. Pat. No. 6,258,812, which is here incorporated by reference. In a particular embodiment, the 4-pyridylmethyl-phthalazine derivative is 1-(4-chloroanilino)-4-(4-pyridylmethyl)-phthalazine or a pharmaceutically acceptable salt thereof. Studies in humans have shown 1-(4-chloroanilino)-4-(4-pyridylmethyl)phthalazine to be well tolerated and to reduce tumor vascular permeability.

Thus, in one aspect, the present invention relates to a method for the prevention of treatment of solid tumors in a mammal, preferably a human patient, which comprises treating the mammal concurrently with a combination of (a) a COX-2 inhibitor of U.S. Pat. No. 6,291,523 and (b) 4-pyridylmethyl-phthalazine VEGF inhibitor of U.S. Pat. No. 6,258,812

In a broader sense of the invention, the term VEGF inhibitor comprises all types of active ingredients, which decrease the activity of the VEGF, and which are especially selected from the group consisting of compounds which inhibit the VEGF receptor tyrosine kinase, compounds which inhibit a VEGF receptor and compounds binding to VEGF. Such an active ingredient, which decreases the activity of the VEGF, is in particular one of those compounds, proteins and monoclonal antibodies, which are generically and specifically disclosed in WO 98/35958, WO 00/09495, WO 00/27820, WO 00/59509, WO 98/11223, WO 00/27819, WO 01/55114, WO 01/58899 and EP 0 769 947, which are described by M. Prewett et al in Cancer Research 59 (1999) 5209-5218, by F. Yuan et al in Proc. Natl. Acad. Sci. USA, vol. 93, pp. 14765-14770, December 1996, by Z. Zhu et al in Cancer Res. 58, 1998, 3209-3214, and by J. Mordenti et al in Toxicologic Pathology, Vol. 27, no. 1, pp 14-21, 1999, those which are generically and specifically disclosed in WO 00/37502 and WO 94/10202; and those which are described by M. S. O'Reilly et al, Cell 79, 1994, 315-328 (Angiostatin™) and by M. S. O'Reilly et al, Cell 88, 1997, 277-285 (Endostatin™), in each case in particular in the pharmaceutical preparations and the final products of the working examples, which are hereby incorporated into the present application by reference to this publications. Comprised are likewise the corresponding stereoisomers as well as the corresponding crystal modifications, e.g. solvates and polymorphs, which are disclosed therein. The compounds used as active ingredients in the combinations disclosed herein can be prepared and administered as described in the cited documents, respectively.

In one aspect, treatment using a COX-2 inhibitor within one of the preferred classes disclosed in U.S. Pat. No. 6,291,523 is combined with treatment using an VEGF inhibitor within one of the preferred classes disclosed in U.S. Pat. No. 6,258,812.

In another aspect, treatment using a COX-2 inhibitor within one of the preferred classes disclosed in U.S. Pat. No. 6,291,523 is combined with treatment using an VEGF inhibitor selected from the group consisting of 1-(4-chloroanilino)-4-(4-pyridylmethyl)phthalazine, or a pharmaceutically acceptable salt thereof.

In a different aspect, treatment using a COX-2 inhibitor selected from the group consisting of 5-methyl-2-(2′-chloro-6′-fluoro-anilino)-phenyl acetic acid, or a pharmaceutically acceptable salt thereof, is combined with treatment using an VEGF inhibitor within one of the preferred classes disclosed in U.S. Pat. No. 6,258,812.

In one aspect, the present invention relates to a method for the prevention or treatment of pre-malignant colon lesions or a colon cancer, as well as other malignancies, in a mammalian patient, especially a human patient, which comprises treating the patient concurrently with a combination of (a) a COX-2 inhibitor selected from the group consisting of 5-methyl-2-(2′-chloro-6′-fluoro-anilino)-phenyl acetic acid, or a pharmaceutically acceptable salt thereof, and (b) an VEGF inhibitor selected from the group consisting of 1-(4-chloroanilino)-4-(4-pyridylmethyl)phthalazine, or a pharmaceutically acceptable salt thereof. In a specific embodiment, the inventive method is a method for the prevention or treatment of colon cancer. In another embodiment, the inventive method is a method for the prevention or treatment of pre-malignant colon lesions.

In addition to the prevention and treatment of pre-malignant colon lesions (e.g. polyps) and colon cancer, the inventive combination therapy has utility for the treatment of “other malignancies”, which is hereby defined as a malignancy that is susceptible to treatment with an VEGF inhibitor, for example, breast cancer, lung cancer, ovarian cancer, lymphoma, head and neck cancer and cancer of the esophagus, stomach, bladder, prostate, uterus and cervix, especially prostate cancer.

According to the present invention, a patient is treated concurrently with a COX-2 inhibitor and an VEGF inhibitor in order to prevent or treat pre-malignant colon lesions, such as polyps, or colon cancer, or another malignancy, each according to a dosage schedule that is appropriate for the individual agent. For example, the COX-2 inhibitor may be administered once or more daily and the VEGF inhibitor may be administered once daily, on alternate days or on some other schedule.

The structure of the active agents identified by code nos., generic or trade names may be taken from the actual edition of the standard compendium “The Merck Index” or from databases, e.g. Patents International (e.g. IMS World Publications). The corresponding content thereof is hereby incorporated by reference.

The present invention further relates to “a combined preparation”, which, as used herein, defines especially a “kit of parts” in the sense that the combination partners (a) and (b) as defined above can be dosed independently or by use of different fixed combinations with distinguished amounts of the combination partners (a) and (b), i.e., simultaneously or at different time points. The parts of the kit of parts can then, e.g., be administered simultaneously or chronologically staggered, that is at different time points and with equal or different time intervals for any part of the kit of parts. The ratio of the total amounts of the combination partner (a) to the combination partner (b) to be administered in the combined preparation can be varied, e.g. in order to cope with the needs of a patient sub-population to be treated or the needs of the single patient based on the severity of the diarrhea that the patient experiences.

The present invention especially relates to a combined preparation, in particular wherein the unit dosage forms are for oral administration, which comprises (a) one or more unit dosage forms of a COX-2 inhibitor and (b) one or more unit dosage forms of an MIA. The present invention especially relates to a combined preparation, which comprises (a) one or more unit dosage forms of a COX-2 inhibitor selected from the group consisting of 5-methyl-2-(2′-chloro-6′-fluoro-anilino)-phenyl acetic acid, or a pharmaceutically acceptable salt thereof, and (b) one or more unit dosage forms of an MIA, especially selected from the group consisting of colchicine, a podophyllotoxin, a taxane, a discodermolide compound, a vinca alkaloid and an epothilone. Preferably, the MIA is a taxane, an epothilone or a discodermolide compound, more preferably the MIA is paclitaxel, docetaxel, epothilone B or (+)-discodermolide, most preferably the MIA is (+)-discodermolide.

Furthermore, the present invention especially relates to a combined preparation, which comprises (a) one or more unit dosage forms of a COX-2 inhibitor and (b) one or more unit dosage forms of an EGFR inhibitor. The present invention especially relates to a combined preparation, which comprises (a) one or more unit dosage forms of a COX-2 inhibitor selected from the group consisting of 5-methyl-2-(2′-chloro-6′-fluoro-anilino)-phenyl acetic acid, or a pharmaceutically acceptable salt thereof, and (b) one or more unit dosage forms of an EGFR inhibitor selected from the group consisting of (R)-4-(4-((1-phenylethyl)amino)-7H-pyrrolo(2,3-d)pyrimidin-6-yl)-phenol, or a pharmaceutically acceptable salt thereof.

Additionally, the present invention relates to a combined preparation which comprises (a) one or more unit dosage forms of a COX-2 inhibitor selected from the group consisting of 5-methyl-2-(2′-chloro-6′-fluoro-anilino)-phenyl acetic acid, or a pharmaceutically acceptable salt thereof, and (b) one or more unit dosage forms of an VEGF inhibitor selected from the group consisting of 1-(4-chloroanilino)-4-(4-pyridylmethyl)phthalazine, or a pharmaceutically acceptable salt thereof, especially, a combined preparation wherein the unit dosage forms are for oral administration.

The combination partner (a) or (b) or a pharmaceutically acceptable salt thereof may also be used in form of a hydrate or other solvate.

A combination which comprises a combination which comprises (a) a selective cyclooxygenase-2 inhibitor of the formula (I) wherein R is methyl or ethyl; R₁ is chloro or fluoro; R₂ is hydrogen or fluoro; R₃ is hydrogen, fluoro, chloro, methyl, ethyl, methoxy, ethoxy or hydroxy; R₄ is hydrogen or fluoro; and R₅ is chloro, fluoro, trifluoromethyl or methyl; pharmaceutically acceptable salts or solvates thereof; and pharmaceutically acceptable prodrug esters thereof; and (b) at least one compound selected from the group consisting of an MIA, an EGFR inhibitor and a VEGF inhibitor, in which the active ingredients (a) and (b) are present in each case in free form or in the form of a pharmaceutically acceptable salt and optionally at least one pharmaceutically acceptable carrier, will be referred to hereinafter as a COMBINATION OF THE INVENTION.

Suitable clinical studies are in particular randomized, double-blind, placebo-controlled, parallel studies in cancer patients with late stage disease, e.g. colon cancer. Such studies are, in particular, suitable to compare the effects of a monotherapy using the active ingredients and a therapy using a COMBINATION OF THE INVENTION, and to prove in particular the synergism of the active ingredients of the COMBINATIONS OF THE INVENTION. The primary endpoints in such studies can be the effect on pain scores, analgesic use, performance status, Quality of Life scores or time to progression of the disease. The radiologic evaluation of tumors in regular time periods, e.g. every 4, 6, 8 or 10 weeks, is a suitable approach to determine the effect of the COMBINATION OF THE INVENTION. In a suitable study design, patients are, for example, randomized in a double-blind fashion receiving a fixed dosage of a COX-2 inhibitor or a corresponding placebo in addition to treatment cycles employing a compound of formula II, e.g. epothilone B, wherein each cycle consists of 0.5, 1.0, 1.5, 2.0 or 2.5 mg/m² epothilone B administered as a 5 minute bolus injection once a week for three weeks followed by one week of rest. Alternatively, the compound of formula II can be administered once every three weeks. The minimum duration of such a study should be about 8 weeks.

It is one objective of this invention to provide a pharmaceutical composition comprising a quantity, which is therapeutically effective pre-malignant colon lesions or colon cancer or other malignancy comprising the COMBINATION OF THE INVENTION. In this composition, the combination partners (a) and (b) can be administered together, one after the other or separately in one combined unit dosage form or in two separate unit dosage forms. The unit dosage form may also be a fixed combination.

When the combination partners employed in the COMBINATION OF THE INVENTION are applied in the form as marketed as single drugs, their dosage and mode of administration can take place in accordance with the information provided on the package insert of the respective marketed drug in order to result in the beneficial effect described herein, if not mentioned herein otherwise.

The effective dosage of each of the combination partners employed in the COMBINATION OF THE INVENTION may vary depending on the particular compound or pharmaceutical composition employed, the mode of administration, the condition being treated, the severity of the condition being treated. Thus, the dosage regimen the COMBINATION OF THE INVENTION is selected in accordance with a variety of factors including the route of administration and the renal and hepatic function of the patient. A physician, clinician or veterinarian of ordinary skill can readily determine and prescribe the effective amount of the single active ingredients required to prevent, counter or arrest the progress of the condition. Optimal precision in achieving concentration of the active ingredients within the range that yields efficacy without toxicity requires a regimen based on the kinetics of the active ingredients' availability to target sites.

In particular, the present invention provides a commercial package comprising (a) a selective cyclooxygenase-2 inhibitor of the formula (I) wherein the radicals and symbols have the meaning as provided above and (b) at least one compound selected from the group consisting of a MIA, a non-covalent EGFR inhibitor and a VEGF inhibitor, together with instructions for simultaneous, separate or sequential use thereof in the treatment of a proliferative disease.

In the instance where the COX-2 inhibitor is 5-methyl-2-(2′-chloro-6′-fluoro-anilino)-phenyl acetic acid, or a pharmaceutically acceptable salt thereof, an appropriate dose is in the range from 100 to 1500 mg of 5-methyl-2-(2′-chloro-6′-fluoro-anilino)-phenyl acetic acid daily, for example, 200-1000 mg/day, such as 200, 400, 500, 600, 800, 900 or 1000 mg/day, administered in one or two doses daily. Preferably, 5-methyl-2-(2′-chloro-6′-fluoro-anilino)-phenyl acetic acid, or a pharmaceutically acceptable salt thereof is administered as an oral pharmaceutal formulation in the form of a tablet, capsule or syrup.

In the instance where the EGFR inhibitor is (R)-4-(4-((1-phenylethyl)amino)-7H-pyrrolo(2,3-d)pyrimidin-6-yl)-phenol, or a pharmaceutically acceptable salt thereof, a daily dose of (R)-4-(4-((1-phenylethyl)amino)-7H-pyrrolo(2,3-d)pyrimidin-6-yl)-phenol in the range from 50 to 2000 mg, for example 1000 mg, 1200 mg, 1500 mg and 2000 mg, is appropriate. It is also possible to administer the above described daily dose efficaciously on a less than daily basis in order to reduce side effects, such as liver toxicity. For example, it is appropriate to administer (R)-4-(4-((1-phenylethyl)amino)-7H-pyrrolo(2,3-d)pyrimidin-6-yl)-phenol, or a pharmaceutically acceptable salt thereof, according to a treatment regimen whereby over at least a three week period, the EGFR inhibitor is administered on only about 40% to about 71% of the days. For example, (R)-4-(4-((1-phenylethyl)amino)-7H-pyrrolo(2,3-d)pyrimidin-6-yl)-phenol, or a pharmaceutically acceptable salt thereof, is administered to the patient from three to five times in each seven day period for a period of three weeks or longer, such as three or four times a week on alternate days for a period of three weeks or longer or three times each week on alternate days, for example, on Monday, Wednesday and Friday of each week, for at least three weeks. The dosage regimen is carried out through at least three or more weeks, for example for 3, 4, 5, 6, 7 or 8 weeks.

In the instance where the VEGF inhibitor is 1-(4-chloroanilino)-4-(4-pyridylmethyl)-phthalazine, or a pharmaceutically acceptable salt thereof, a daily dose of 1-(4-chloroanilino)-4-(4-pyridylmethyl)phthalazine in the range from 50 to 2000 mg, for example 1000 mg, 1200 mg, 1500 mg and 2000 mg, is appropriate. It is also possible to administer the above described daily dose efficaciously on a less than daily basis in order to reduce side effects, such as liver toxicity. For example, it is appropriate to administer 1-(4-chloroanilino)-4-(4-pyridylmethyl)phthalazine, or a pharmaceutically acceptable salt thereof, according to a treatment regimen whereby over at least a three week period, the VEGF inhibitor is administered on only about 40% to about 71% of the days. For example, 1-(4-chloroanilino)-4-(4-pyridylmethyl)phthalazine, or a pharmaceutically acceptable salt thereof, is administered to the patient from three to five times in each seven day period for a period of three weeks or longer, such as three or four times a week on alternate days for a period of three weeks or longer or three times each week on alternate days, for example, on Monday, Wednesday and Friday of each week, for at least three weeks. The dosage regimen is carried out through at least three or more weeks, for example for 3, 4, 5, 6, 7 or 8 weeks.

As used herein, the expression “week” means seven consecutive days. Thus, a three week period is twenty-one consecutive days starting on any day of the calendar week. The day that the first dose is given is considered to be the first day of the week. Any discussion using calendar weeks is intended to be for illustrative purposes only.

Preferably, (R)-4-(4-((1-phenylethyl)amino)-7H-pyrrolo(2,3-d)pyrimidin-6-yl)-phenol, or a pharmaceutically acceptable salt thereof, or a pharmaceutically acceptable salt thereof, is administered as an oral pharmaceutal formulation in the form of a tablet, capsule or syrup.

Preferably, 1-(4-chloroanilino)-4-(4-pyridylmethyl)phthalazine, or a pharmaceutically acceptable salt thereof, or a pharmaceutically acceptable salt thereof, is administered as an oral pharmaceutal formulation in the form of a tablet, capsule or syrup.

Epothilone B is preferably administered in a dose which is calculated according to the formula (II)

single dose (mg/m2)=(0.1 to y)×N (III)

wherein N is the number of weeks between treatments and y is 6, wherein epothilone B is administered in more than one treatment cycle after an interval of one week to six weeks after the preceding treatment.

In one preferred embodiment of the invention, epothilone B is administered weekly in a dose that is between about 0.1 to 6 mg/m², preferably between 0.1 and 3 mg/m², e.g. 2.5 or 3.0 mg/m², for three weeks after an interval of one to six weeks, especially an interval of one week, after the preceding treatment. In another embodiment of the invention said epothilone B is preferably administered to a human every 18 to 24 days in a dose that is between about 0.3 and 12 mg/m².

Etoposide phosphate may be administered to a human in a dosage range varying from about 25 to 115 mg/m² day, e.g. 56.8 or 113.6 mg/m² day.

Teniposide may be administered to a human in a dosage range varying from about 75 to 150 mg about every two weeks.

Paclitaxel may be administered to a human in a dosage range varying from about 50 to 300 mg/m² day.

Vinblastine may be administered to a human in a dosage range varying from about 1.5 to 10 mg/m² day.

Vincristine sulfate may be administered parenterally to a human in a dosage range varying from about 0.025 to 0.05 mg/kg body weight*week.

Vinorelbine may be administered to a human in a dosage range varying from about 10 to 50 mg/m² day.

Furthermore, the present invention pertains to the use of a combination which comprises (a) a selective cyclooxygenase-2 inhibitor, in particular a selective cyclooxygenase-2 inhibitor of the formula (I) wherein the radicals and symbols have the meanings as provided above or a pharmaceutically acceptable prodrug ester thereof, and (b) a vascular endothelial growth factor receptor tyrosine kinase inhibitor, in which the active ingredients (a) and (b) are present in each case in free form or in the form of a pharmaceutically acceptable salt and optionally at least one pharmaceutically acceptable carrier; for the preparation of a medicament for the treatment of a proliferative disease, especially for the treatment of cancer of the prostate.

Moreover, the present invention pertains to the use of a combination which comprises (a) a selective cyclooxygenase-2 inhibitor, in particular a selective cyclooxygenase-2 inhibitor of the formula (I) wherein the radicals and symbols have the meanings as provided above or a pharmaceutically acceptable prodrug ester thereof, and (b) at least one compound selected from the group consisting of a microtubule interfering agent, a non-covalent epithelial growth factor receptor tyrosine protein kinase inhibitor and a vascular endothelial growth factor receptor tyrosine kinase inhibitor, in which the active ingredients (a) and (b) are present in each case in free form or in the form of a pharmaceutically acceptable salt and optionally at least one pharmaceutically acceptable carrier; for the preparation of a medicament for the treatment of a proliferative disease, in particular for the prevention or treatment of pre-malignant colon lesions or colon cancer.

The following Examples illustrate the invention described above; they are not, however, intended to limit the scope of the invention in any way. The beneficial effects of the COMBINATION OF THE INVENTION can also be determined by other test models known as such to the person skilled in the pertinent art.

### EXAMPLE 1

5-methyl-2-(2′-chloro-6′-fluoro-anilino)-phenyl acetic acid (“COX”) and (+)-discodermolide (“disco”) are tested as single agents and together as combination therapy in a mouse model of adenomatous polyposis for the prevention and treatment of intestinal polyps. (+)-Discodermolide is administered once intravenously to the mice at 15 mg/kg as a solution in 16.7% Chremophor EI, 8.3% ethanol, and 75% D5W. COX is administered in the feed mix at a concentration of 125 ppm. The following results of duplicate experiments are observed:

| | | | | POLYPS | | | |
|---|---|---|---|---|---|---|---|
| | | | | Mean | | ANIMALS | |
| DRUGS | | | | Intestinal | | % Body | |
| Com- | | | Dose | Polyp Count | % | Wt. | Dead/ |
| pound | Route | Regimen | (mg/kg) | (# ± SEM) | T/C | Change | Total |
| Control | feed | ad libitum | — | 28 ± 1.6 | — | +8.2 ± 0.7 | 0/3 |
| COX | feed | ad libitum | 125 ppm | 15 ± 0.4 | 56 | +3.4 ± 0.1 | 0/7 |
| disco | i.v. | Once | 15 mg/kg | 15 ± 0.2 | 56 | +0.9 ± 0.1 | 0/7 |
| COX + | feed + | ad libitum + | 125 ppm + 15 mg/kg | 8 ± 0.3 | 29 | −5.0 ± 0.1 | 0/7 |
| disco | i.v. | Once | | | | | |
| Control | feed | ad libitum | — | 23 ± 0.69 | — | +18.5 ± 0.21 | 0/4 |
| COX | feed | ad libitum | 125 ppm | 13 ± 0.2 | 57 | +14.9 ± 0.14 | 0/7 |
| disco | i.v. | Once | 15 mg/kg | 15 ± 0.22 | 64 | +4.0 ± 0.12 | 0/7 |
| COX + | feed + | ad libitum + | 125 ppm + 15 mg/kg | 8 ± 0.17 | 34 | −3.4 ± 0.11 | 0/7 |
| disco | i.v. | Once | | | | | |

Both agents alone cause a statistically significant reduction in the number of newly formed intestinal polyps. The combination further reduces the number of new polyps to a level that is lower than either agent alone and that is statistically significant. Statistical evaluations are performed using a one tailed Student t-test and all p values are less than 0.01.

### EXAMPLE 2

5-methyl-2-(2′-chloro-6′-fluoro-anilino)-phenyl acetic acid (“COX”) and (R)-4-(4-((1-phenylethyl)amino)-7H-pyrrolo(2,3-d)pyrimidin-6-yl)-phenol (“EGFR”) are tested as single agents and together as combination therapy in a mouse model of adenomatous polyposis for the prevention and treatment of intestinal polyps. EGFR is administered to the mice orally at 50 mg/kg, as a suspension in 0.5% carboxymethylcellulose, b.i.d., 5 days a week for three weeks. COX is administered in the feed mix at a concentration of 125 ppm. The following results are observed in duplicate experiments:

| | | | | POLYPS | | | |
|---|---|---|---|---|---|---|---|
| | | | | Mean | | ANIMALS | |
| DRUGS | | | | Intestinal | | % Body | |
| Com- | | | Dose | Polyp Count | % | Wt. | Dead/ |
| pound | Route | Regimen | (mg/kg) | (# ± SEM) | T/C | Change | Total |
| Control | feed | ad libitum | — | 22 ± 0.4 | — | 8.2 ± 0.1 | 0/4 |
| COX | feed | ad libitum | 125 ppm | 9 ± 0.4 | 43 | 3.5 ± 0.1 | 0/7 |
| EGFR | p.o., | 5x/week | 50 mg/kg | 8 ± 0.2 | 37 | −2.8 ± 0.1 | 0/7 |
| | b.i.d. | | | | | | |
| COX + | feed + | ad libitum + | 125 ppm + 50 mg/kg | 5 ± 0.3 | 25 | −0.4 ± 0.1 | 0/7 |
| EGFR | p.o., | 5x/week | | | | | |
| | b.i.d. | | | | | | |
| Control | feed | ad libitum | — | 32 ± 2.5 | — | 5.2 ± 0.3 | 0/4 |
| COX | feed | ad libitum | 125 ppm | 12 ± 0.9 | 36 | 5.5 ± 1.2 | 0/7 |
| EGFR | p.o., | 5x/week | 50 mg/kg | 9 ± 0.3 | 27 | 2.5 ± 0.1 | 0/7 |
| | b.i.d. | | | | | | |
| COX + | feed + | ad libitum + | 125 ppm + 50 mg/kg | 6 ± 0.2 | 18 | −0.7 ± 0.2 | 0/7 |
| EGFR | p.o., | 5x/week | | | | | |
| | b.i.d. | | | | | | |

Both agents alone cause a statistically significant reduction in the number of newly formed intestinal polyps. The combination further reduces the number of new polyps to a level that is lower than either agent alone and that is statistically significant. Statistical evaluations are performed using a one tailed Student t-test and all p values are less than 0.001.

### EXAMPLE 3

5-methyl-2-(2′-chloro-6′-fluoro-anilino)-phenyl acetic acid (“COX”) and 1-(4-chloroanilino)-4-(4-pyridylmethyl)phthalazine (“VEGFR”) are tested as single agents and together as combination therapy in a mouse model of adenomatous polyposis for the prevention and treatment of intestinal polyps. VEGFR is administered to the mice orally at 100 mg/kg, 5 times a week for three weeks. COX is administered in the feed mix at a concentration of 125 ppm. The following results are observed in duplicate experiments:

| | | | | POLYPS | | | |
|---|---|---|---|---|---|---|---|
| | | | | Mean | | ANIMALS | |
| DRUGS | | | | Intestinal | | % Body | |
| Com- | | | Dose | Polyp Count | | Wt. | Dead/ |
| pound | Route | Regimen | (mg/kg) | (# ± SEM) | % T/C | Change | Total |
| Control | feed | ad libitum | — | 27 ± 3.41 | — | 9.17 ± 0.19 | 0/4 |
| COX | feed | ad libitum | 125 ppm | 12 ± 0.22 | 44 | 8.12 ± 0.13 | 0/7 |
| VEGF | p.o. | 5x/week | 100 mg/kg | 15 ± 0.48 | 57 | 5.46 ± 0.24 | 0/6 |
| COX + | feed + | ad libitum + | 125 ppm + 100 mg/kg | 9 ± 0.24 | 33 | 5.31 ± 0.11 | 0/7 |
| VEGF | p.o. | 5x/week | | | | | |
| Control | feed | ad libitum | — | 29 ± 1.78 | — | 2.3 ± 0.27 | 0/4 |
| COX | feed | ad libitum | 125 ppm | 13 ± 0.18 | 44 | 0.9 ± 0.09 | 0/7 |
| VEGF | p.o. | 5x/week | 100 mg/kg | 17 ± 0.19 | 60 | 1.7 ± 0.13 | 0/7 |
| COX + | feed + | ad libitum + | 125 ppm + 100 mg/kg | 8 ± 0.19 | 29 | 1.0 ± 0.13 | 0/7 |
| VEGF | p.o. | 5x/week | | | | | |

Both agents alone cause a statistically significant reduction in the number of newly formed intestinal polyps. The combination further reduces the number of new polyps to a level that is lower than either agent alone and that is statistically significant. Statistical evaluations are performed using a one tailed Student t-test and all p values are less than 0.001.

### EXAMPLE 4

5-methyl-2-(2′-chloro-6′-fluoro-anilino)-phenyl acetic acid (“COX”) and 1-(4-chloroanilino)-4-(4-pyridylmethyl)phthalazine (“VEGFR”) are tested as single agents and together as combination therapy in a mouse model of prostrate cancer (orthoscopically implanted DU 145 prostate tumor cell line) for efficacy against established tumors. VEGFR is administered to the mice orally at 100 mg/kg, 5 times a week for three weeks, as a suspension in 0.5% microcrystalline cellulose, 0.1% Tween 80 in water. COX is administered in the feed mix at a concentration of 125 ppm. The following results are observed:

| | | | | TUMORS | | | |
|---|---|---|---|---|---|---|---|
| | | | | Mean | | ANIMALS | |
| DRUGS | | | | Tumor | | % Body | |
| Com- | | | Dose | Weight | | Wt. | Dead/ |
| pound | Route | Regimen | (mg/kg) | (mg ± SEM) | % T/C | Change | Total |
| Control | p.o. | q.d. 5x/week | — | 428 ± 69 | — | 3.6 ± 0.72 | 0/12 |
| (CMC/ | | | | | | | |
| Tween/ | | | | | | | |
| H₂0) | | | | | | | |
| COX | feed | powder, daily | 125 ppm | 343 ± 52 | 80 | 4.9 ± 1.28 | 1/12 |
| VEGF | p.o. | q.d. 5x/week | 100 mg/kg | 243 ± 37 | 57 | 9.9 ± 3.39 | 1/12 |
| COX + | feed + | ad libitum + | 125 ppm + 100 mg/kg | 134 ± 27 | 31 | 3.5 ± 2.96 | 0/12 |
| VEGF | p.o. | 5x/week | | | | | |

1-28. (canceled)

29. A combination which comprises a first active ingredient which is a selective cyclooxygenase-2 inhibitor and a second active ingredient which is epothilone B, in which the first and second active ingredients are present in each case in free form or in the form of a pharmaceutically acceptable salt, for simultaneous, separate or sequential use.

30. A method of treating a patient with a condition selected from the group consisting of colon lesions, colon cancer, prostate cancer, breast cancer, lung cancer, ovarian cancer, lymphoma, head and neck cancer, cancer of the esophagus, stomach cancer, bladder cancer, uterine cancer and cervical cancer, which comprises administering to the patient a first active ingredient which is a selective cyclooxygenase-2 inhibitor and a second active ingredient which is epothilone B, in which the first and second active ingredients are present in each case in free form or in the form of a pharmaceutically acceptable salt, and are administered simultaneously, separately or sequentially.

31. A method of claim 30 wherein the condition is colon cancer.
